(19) United States
(12) Patent Application Publication
(10) Pub. No.: US 2005/0277811 A1
(43) Pub. Date: Dec. 15, 2005

(54) Speculum

(75) Inventors: Charlotte Richards, Newton, MA (US); Richard Ross, Newton, MA (US); JoAnn Jorge, Ashland, MA (US); Francis Gleason, Ashland, MA (US); Randal B. Chinnock, Southbridge, MA (US); Ronald Forni, Littleton, MA (US); John W. Langer, Sturbridge, MA (US); John Walsh, Norfolk, MA (US); Brian Anderson, Ashland, MA (US); Ken Doucette, Norton, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Innovative Gynecological Solutions, LLC, Framingham, MA (US)

(21) Appl. No.: 11/137,290

(22) Filed: May 25, 2005

Related U.S. Application Data
(60) Provisional application No. 60/574,045, filed on May 25, 2004.

(51) Int. Cl. A61B 1/00

(52) U.S. Cl. 600/184

(57) ABSTRACT

A speculum for use with a body opening, such as a vagina, rectum or surgical wound, may have an insertion portion with an arcuately-shaped wall and a slot that extends along a length of the wall. An end face of the wall at the distal end of the insertion portion may be arranged at a non-perpendicular angle to the longitudinal axis of the insertion portion. A flange portion joined at a proximal end of the insertion portion may have a continuous band that extends radially outwardly from the wall and includes a notch that communicates with the slot. A handle portion may have a gripping surface extending proximally at a bottom side of the flange portion with an arcuate shape that is approximately concentric with the wall of the insertion portion. A top side of the flange portion may be free of the handle portion.

### RELATED APPLICATIONS

This application claims the benefit of U.S. Provisional Application 60/574,045, filed May 25, 2004, which is hereby incorporated by reference in its entirety.

### BACKGROUND OF INVENTION

1. Field of the Invention

The present invention relates to specula, including a speculum for use in accessing an opening in a body, such as a vagina or a surgical wound.

2. Related Art

Specula are commonly used to access openings in a body, such as a vagina, ear canal, mouth, rectum, etc. One speculum configuration used with vaginas is a bivalve instrument having two blades that are movable relative to each other. The speculum is placed in the vagina with the blades collapsed together, and is advanced in different directions, e.g., toward the patient's abdomen and her back. Once the cervix is located, which in some individuals can be difficult due to anatomical variations of the cervix, body weight of the patient, laxity of the vaginal wall secondary to childbirth, height of the patient, and/or anxiety and increased muscle tone of the patient, one blade of the speculum is used to put traction on either the ventral aspect of the cervix (the part nearest the patient's abdomen), or on the dorsal aspect of the cervix (the part nearest the patient's back). This motion involves opening and closing the blades of the speculum, as well as pulling it back and forth, in and out of the vagina. The sensation of ‘digging’, moving, stretching, with an oftentimes cold speculum may be uncomfortable and/or embarrassing for the patient. Sometimes an examination performed with such a device is accompanied by tearing of the vaginal skin, pinching of the vulva (the outside part of the vagina), and/or bruising or bleeding of the cervix and vagina.

### SUMMARY OF INVENTION

In one aspect of the invention, a speculum is provided with no blades or other parts that are required to be moved to access a body opening. In one illustrative embodiment, a speculum includes an insertion portion having a barrel-like shape that may be inserted into the body opening with or without an obturator. Once positioned in the opening, the insertion portion may provide access to the opening via a passageway at the interior of the insertion portion. The passageway may be formed by an arcuate-shaped wall that extends from a proximal end to a distal end of the insertion portion. The wall may include a slot or other opening that extends along a portion of the length of the insertion portion, or that extends along an entire length of the insertion portion. The insertion portion may also have a beveled end such that an end face of the wall at the distal end of the insertion portion is arranged at an angle to a longitudinal axis of the insertion portion, or otherwise be arranged so that one section of the distal end of the insertion portion extends distally further than other sections of the insertion portion.

In one aspect of the invention, the speculum may include a flange portion at a proximal end of the insertion portion. The flange portion may have a continuous band section that extends radially away from the insertion portion, e.g., to limit the insertion depth of the device and/or help protect hair or other body features near the entrance of the body opening. The band section may have a notch or other opening that does not break the continuity of the band, but communicates with a slot in the insertion portion, e.g., to allow operation of a surgical instrument at least partially disposed in the passageway.

In another aspect of the invention, the speculum may include an illumination portion that provides light to at least a part of the insertion portion. The light may be transmitted toward the distal end of the insertion portion through the insertion portion and/or through the passageway. Different color lights may be used, e.g., to provide different illumination to help a physician detect different medical conditions, as is the case, for example, with the use of green light and vinegar in some vaginal examinations. The illumination may be therapeutic, e.g., light that kills or inhibits the growth of particular microbes or otherwise treats a condition. The illumination portion may provide light to the insertion portion of the speculum and/or an obturator (if used). The illumination portion may be made removably attachable to the speculum, e.g., to the flange portion or obturator.

In one aspect of the invention, a flange portion of the speculum may include a recess that receives an illumination portion. The recess may be located at a bottom side of the flange portion and be arcuately shaped. This arrangement may provide for a relatively obstruction-free upper side of the flange portion, allowing easier access to the passageway for viewing purposes, insertion of a surgical instrument, or other. In another aspect of the invention, the flange portion may include an extension that communicates with the passageway and provides a path for fluid in the passageway to exit the passageway while avoiding contact with the illumination portion.

In one aspect of the invention, a handle portion or other gripping portion may have a circular or arcuate shape that extends around a portion of the speculum passageway. The handle portion may extend radially no further than other parts of the speculum so as to provide a compact device that provides for 360 degree rotation about a longitudinal axis of the passageway without interference with a patient's legs or other body parts near or around the body opening.

In one aspect of the invention, a medical instrument for use in accessing an opening in a body is provided having an insertion portion constructed and arranged for insertion into the opening in the body. The insertion portion may have an arcuate-shaped wall that extends from a proximal end to a distal end of the insertion portion, and defines an elongated passageway from the proximal end to the distal end. A slot may extend along the entire length of the wall from the proximal end to the distal end. A flange portion having a section that forms a continuous band may be joined to the wall at the proximal end of the insertion portion and extends radially outwardly away from the passageway. A notch in the flange portion may communicate with the slot in the wall of the insertion portion. The insertion portion may be sized to be inserted into the opening in the body with the flange portion positioned outside of the opening so that the insertion portion displaces at least some of the body material, such as tissue, at the opening and the passageway in the insertion portion provides access to the opening.

In another aspect of the invention, a medical instrument for use in accessing an opening in a body is provided having an insertion portion constructed and arranged for insertion into the opening in the body. The insertion portion may have an arcuately-shaped wall that extends from a proximal end to a distal end of the insertion portion so the wall defines an elongated passageway having a longitudinal axis extending from the proximal end to the distal end. A slot may extend along an entire length of the wall from the proximal end to the distal end, and an end face of the wall at the distal end of the insertion portion may be arranged at a non-perpendicular angle to the longitudinal axis. A flange portion may have a portion that is joined to the wall at the proximal end of the insertion portion and that extends radially outwardly away from the passageway. The insertion portion may be sized to be inserted into the opening in the body and displace at least some of the body material with the flange portion positioned outside of the opening so that the elongated passageway provides access to the opening.

In another aspect of the invention, a medical instrument for use in accessing an opening in a body is provided having an insertion portion constructed and arranged for insertion into an opening in a body. The insertion portion may have a wall with an approximately arcuate crosssection that extends from a proximal end to a distal end of the insertion portion so that the wall defines an elongated passageway having a longitudinal axis. A flange portion may have a portion that is joined to the wall at the proximal end of the insertion portion and that extends radially outwardly away from the passageway. A handle portion with a gripping surface adapted to be gripped by a user for manipulation of the speculum may extend proximally at a bottom side of the flange portion and have an arcuate shape that is approximately concentric with the wall of the insertion portion. A top side of the flange portion may be free of the handle portion so as to permit access to the passageway.

In one embodiment, the speculum may be arranged so that the wall forms an approximately cylindrically-shaped barrel having a longitudinal axis, and a slot formed in the wall extends along the longitudinal axis.

In another embodiment, the speculum may include an obturator constructed and arranged to be inserted into the passageway of the insertion portion. The obturator may have a distal end constructed and arranged to extend from the distal end of the insertion portion and facilitate insertion of the insertion portion into the opening in the body.

In another embodiment, the speculum may include an illumination portion arranged to provide light to the insertion portion. For example, the illumination portion may provide light to two or more separate portions of the insertion portion so the light is directed toward a distal end of the insertion portion.

These and other aspects of the invention will be apparent from the following description and claims.

### BRIEF DESCRIPTION OF THE DRAWINGS

Aspects of the invention are described below with reference to the following drawings in which like reference numerals reference like elements, and wherein:

FIG. 1 is an exploded perspective view of an embodiment of a speculum with an optional illumination portion in accordance with the invention.

FIG. 2 is a top view of the FIG. 1 embodiment with an optional obturator.

FIG. 3 is a side view of the FIG. 1 embodiment.

FIG. 4 is a bottom view of the FIG. 1 embodiment.

FIG. 5 is a front view of the FIG. 1 embodiment.

FIG. 6 is a rear perspective view of the FIG. 1 embodiment.

FIG. 7 is a rear view of the FIG. 1 embodiment.

FIG. 8 is a rear perspective view of another illustrative embodiment of a speculum in accordance with the invention.

FIG. 9 is a rear view of the FIG. 8 embodiment.

FIG. 10 is a front view of the FIG. 8 embodiment.

FIG. 11 is a rear perspective view of yet another illustrative embodiment of a speculum in accordance with the invention.

FIG. 12 is a front perspective view of the FIG. 11 embodiment.

FIG. 13 is a side perspective view of the FIG. 11 embodiment.

FIG. 14 is a side perspective view of yet another illustrative embodiment of a speculum in accordance with the invention.

FIG. 15 is a top view of the FIG. 14 embodiment.

FIG. 16 is a bottom view of the FIG. 14 embodiment.

FIG. 17 is a side view of the FIG. 14 embodiment.

FIG. 18 is a front view of the FIG. 14 embodiment.

FIG. 19 is a rear view of the FIG. 14 embodiment.

FIG. 20 is a rear perspective view of the FIG. 14 embodiment.

FIG. 21 is a rear perspective view of yet another illustrative embodiment of a speculum in accordance with the invention.

FIG. 22 is a front view of the FIG. 21 embodiment

FIG. 23 is a rear perspective view of yet another illustrative embodiment of a speculum in accordance with the invention.

FIG. 24 is a front perspective view of the FIG. 23 embodiment.

FIG. 25 is a side perspective view of yet another illustrative embodiment of a speculum in accordance with the invention.

FIG. 26 is a front perspective view of the FIG. 25 embodiment.

FIG. 27 is a rear perspective view of the FIG. 25 embodiment.

### DETAILED DESCRIPTION

Illustrative embodiments of a speculum for gynecological procedures including examinations, screenings, biopsies, and surgeries of the vagina, cervix and uterus are described below. However, aspects of the invention are not limited to the illustrative embodiments described, but rather may be incorporated, whether alone or in combination, with other aspects of the invention, in any suitable arrangement. In addition, aspects of the invention are not limited to use with respect to a vagina, but may be used with any body opening, such as a surgical wound, ear canal, mouth or throat, rectum, etc.

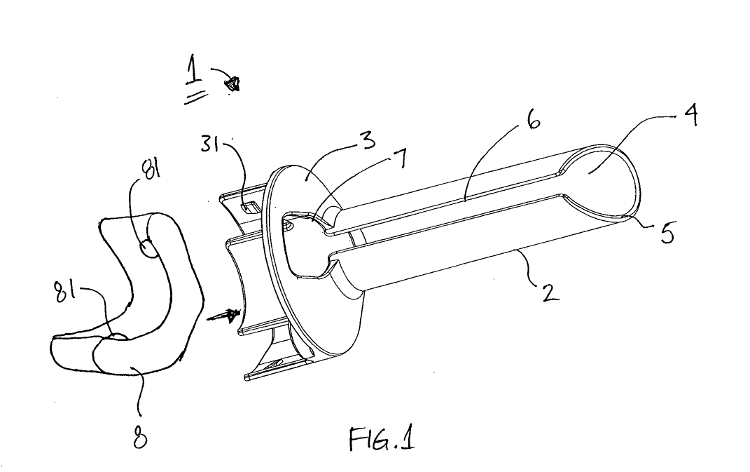

FIG. 1 shows a top-side perspective view of an embodiment of a speculum 1 in accordance with the invention. In this embodiment, the speculum 1 includes an insertion portion 2 that extends from a proximal end near a flange portion 3 to a distal end. The insertion portion 2 includes a wall having an arcuate cross-sectional shape that defines an elongated passageway 4 for the insertion portion 2. As used herein, “arcuate” refers to shapes including a circular shape or portion of a circle, curves having portions with two or more radii (e.g., an oval or a portion of an oval), a shape formed by two or more straight portions (e.g., a polygon or a portion of a polygon), and other irregular shapes that approximate a curve. In this embodiment, the wall of the insertion portion 2 forms a barrel-shaped member that is approximately cylindrical, but the wall may form other suitable shapes, such as a conical, or frustoconical barrel, a tube having an oval, irregular or polygonal cross-sectional shape, and so on. The outer surface of the insertion portion 2 may be smooth, may have longitudinal ribs along the outer surface, or may include other surface features as desired.

In one aspect of the invention, the insertion portion 2 may include a slot 6 that extends along an entire length of the insertion portion 2, e.g., from a junction with the flange portion 3 to the distal end of the insertion portion. The slot 6 may be straight as shown, or may arranged in any other suitable way, such as in a spiral, may have a varying width, and so on. (In some embodiments, the slot 6 may be eliminated.) Edge portions of the slot 6 and the end face 5 may be tapered, rounded or otherwise arranged so as to help avoid catching on tissue or other structures in the opening, such as when inserting the speculum 1 into the opening or rotating the speculum 1. The slot 6 may communicate with a notch 7 or other opening in the flange portion 3. The notch 7 and slot 6 may provide space for surgical instruments, such as a tenaculum, to be passed through the passageway 4 and operated. In addition, the notch 7 may provide a more open area at an upper side of the flange portion 3 so as to allow easier access to the passageway 4 for viewing, instrument insertion, etc. The notch 7 may be formed in a section of the flange portion that forms, in one aspect of the invention, a continuous band, i.e., forms a continuous pathway around the passageway 4 without breaks. The continuous band section may be joined to the wall at the proximal end of the insertion portion 2 and extend radially outwardly away from the passageway 4, e.g., to protect body portions around the opening from contact with surgical instruments and/or limit the insertion depth of the insertion portion 2.

In this illustrative embodiment, the speculum 1 includes an illumination portion 8 that provides light to illuminate an area of interest. In one aspect of the invention, light may be provided so as to illuminate a body portion that is located at the distal end of the insertion portion and/or positioned within the passageway 4. The illumination portion 8 may include one or more light emitting regions 81 that provide light to other portions of the speculum 1, such as the insertion portion 2. The light may be transmitted through the passageway 4 and/or through one or more portions of the insertion portion 2 toward the distal end. Light may be provided by the illumination portion by any suitable means, such as one or more light emitting diodes (LEDs), incandescent lamps, light emitting polymer, and so on that are exposed at or otherwise provide light to light emitting regions 81.

The illumination portion 8 may be made disposable, reusable and/or sterilizable, and may include its own power supply, e.g., rechargeable batteries, or may be connectable to an external power source. The illumination portion 8 may have a sealed housing, e.g., include sealed plastic walls to prevent liquids from entering the housing, and/or may be made so that the housing may be opened to allow repair, battery replacement, etc. and closed to reseal the housing. The illumination portion 8 may include a manually actuated switch to turn the light source(s) on/off, and/or may include a device that enables the illumination portion 8 to be turned on when attached to the speculum 1, but otherwise disables the illumination portion 8. For example, the illumination portion 8 may include a switch that is closed when the illumination portion is connected to the speculum 1, but is open when the illumination portion is separated from the speculum. This may help avoid inadvertently leaving the illumination portion on when not in use. The illumination portion 8 may also include a “dimming” feature or other arrangement to control the intensity or other features of the light emitted. The illumination portion 8 may also include a red indicator light or other display that indicates a low battery condition, illumination malfunction or other information.

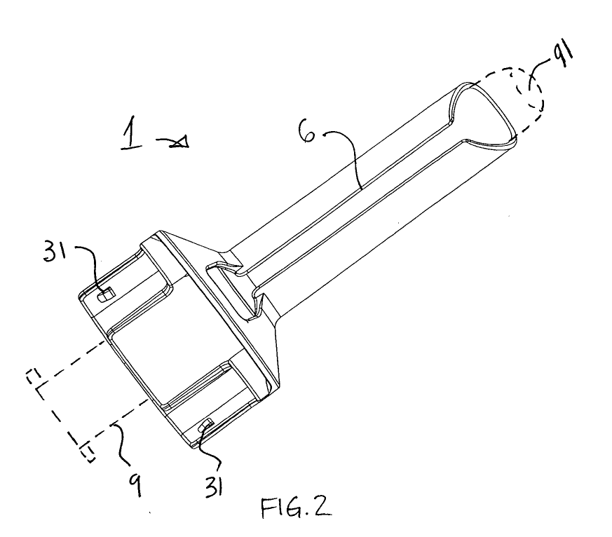

In one aspect of the invention, the illumination portion 8 may be arranged at a bottom side of the speculum 1, e.g., positioned in a recess in the flange portion 3, so as to provide a relatively obstruction-free upper side at the flange portion 3 to allow easier access to the passageway 4. In accordance with this aspect of the invention, the illumination portion 8 may be removably attached to the speculum 1, as shown. FIG. 2 shows a pair of tabs 31 that may be used to engage the illumination portion 8 and help retain the illumination portion in the recess of the flange portion 3. The tabs 31 may resiliently engage with grooves or notches formed in the housing of the illumination portion 8 so that the illumination portion is securely held in place during use, but may be relatively easily removed, e.g., after use when discarding the insertion portion 2. Of course, it will be understood that other features may be used to engage the illumination portion with other parts of the speculum 1, such as threaded fasteners, interference or friction fit features, or other.

In another embodiment, the illumination portion may be at least partially integrated into other parts of the speculum 1, such as the insertion portion 2. For example, the illumination portion may include one or more LEDs that are imbedded in the insertion portion. A battery or other power source may also be imbedded in or otherwise attached to the speculum and provide power to the LEDs. Alternately, the LEDs may receive electrical energy via wires that run to an external power source separate from the speculum. The illumination portion may alternately include light emitting polymer sections that are suitably positioned in or on the speculum and emit light in a desired manner. Other illumination arrangements will be appreciated by those of skill in the art.

Light provided by the illumination portion 8 may be transmitted by light pipes or other structures that are attached to, or otherwise incorporated into the speculum. As will be discussed below in more detail, the insertion portion 2 may be made of a transparent or translucent material that transmits light from the illumination portion to an area of interest. In addition to enabling the transmission of light, transparent sections of the insertion portion 2 may allow a user to view features of the body opening through the wall of the insertion portion 2. Clear lubricant or other liquid material may aid in viewing such features. Light pipes or other structures that transmit light may be formed by structural features of the wall itself, may be formed by separate structures (e.g., one or more rods) that are molded into or attached to the wall of the insertion portion, or may be removably positioned in the passageway. Lens, diffuser, or other optical features incorporated with the insertion portion 2 may serve to direct light and uniformly illuminate the area of interest.

As shown in a top view in FIG. 2, the speculum 1 may include an obturator 9 or other device to aid in the insertion of the speculum 1 or provide other functions. Alternatively, the insertion portion 2 may be inserted into an opening without the use of an obturator 9 or similar device. For example, other embodiments of the insertion portion 2 may obviate the need for an obturator 9, such as an arrangement in which the insertion portion 2 includes a feature such as a retractable rounded end that may overlie the distal end during insertion of the speculum, and then be retracted. In such an embodiment, the insertion portion may both facilitate speculum insertion and the conduct of the examination after insertion. The obturator 9 may have an external shape that closely conforms to the inner surface of the wall in the passageway 4, e.g., have a cylindrical portion that extends into the passageway as shown. In one embodiment, the obturator 9 may include a key, raised rib or other feature that engages with the slot 6. This feature may define a particular rotational position of the obturator 9 relative to the insertion portion 2 and/or serve to at least partially fill the space at the slot 6 so as to aid in insertion or rotation of the insertion portion 2 in the opening. The obturator 9 may have a blunt, tapered, cone-shaped or otherwise suitably formed distal end that is carried on a slender rod that extends proximally to allow manipulation by the user. The obturator 9 may have an opening 91 at a distal end that is suitable for performing certain functions, such as to visualize and/or perform procedures on the cervical os, fetal scalp, or other. For example, the distal end of the obturator 9 around the opening 91 may be positioned against the fetal scalp, and the scalp may be accessed for certain examinations. The opening 91 may also be used to guide instruments into the uterus, e.g., for endometrial biopsy, examination of the fetal head (for obtaining scalp pH), embryo transfer, endocervical curettage, and hysteroscopy.

The obturator 9 may also be used to stop uterine bleeding from the cervix, and may be made of a clear or transparent material (e.g., plastic). The obturator 9 (or the insertion portion 2) may also be formed of suitable color, transparent materials to enhance visualization and diagnosis of certain conditions, such as cervicitis, dysplasia, inflammation, developmental anomalies, hypervascularity, lacerations, bruises and discharges, etc. As discussed above, the obturator 9 may transmit illumination provided by an illumination portion 8, whether for therapeutic or visualization purposes. The obturator 9 may include a stop, ring or other feature to limit the insertion depth of obturator into the passageway 4, or may be arranged so as to pass entirely through the passageway 4.

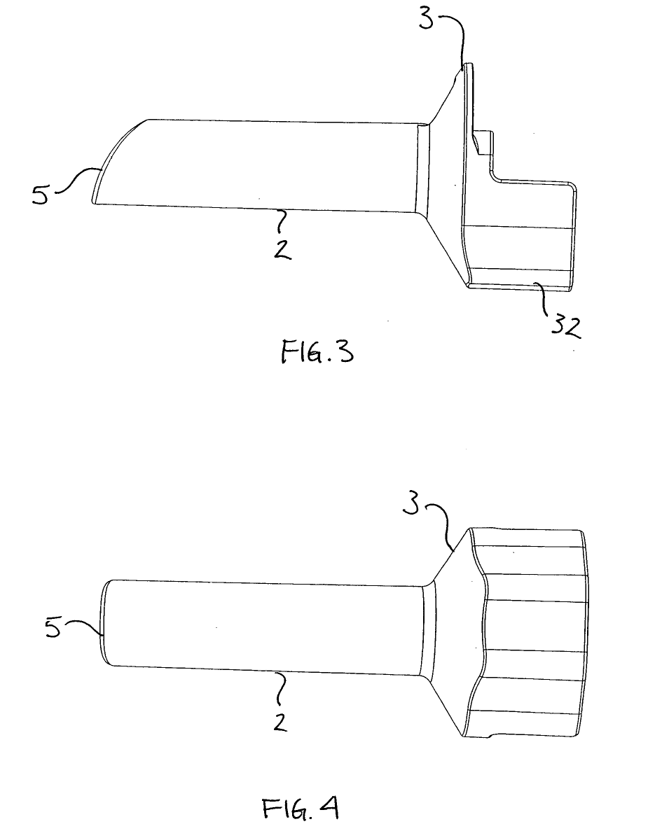

In one aspect of the invention, one portion of the distal end of the insertion portion 2 may extend distally further than other portions of the distal end. For example, an end face 5 of the wall of the insertion member may beveled, i.e., be arranged at an angle to a longitudinal axis of the passageway 4. This arrangement results in one portion of the end face 5 extending distally farther from the flange portion 3 than another portion of the end face 5. This arrangement may allow a user of the speculum 1 to manipulate portions of the body in or near the opening, such as a portion of a cervix, by contacting the body portion with the extended section of the insertion portion 2, or allow for better viewing or access. For example, as shown in a side view of the speculum 1 in FIG. 3, the end face 5 of the insertion portion 2 may be arranged at an angle of approximately 45 degrees to a longitudinal axis of the passageway 4. In this embodiment, the bottom section of the insertion portion 2, i.e., the section opposite the slot 6, is somewhat longer than the upper section near the slot 6. Of course, it will be understood that the beveled end, if provided, may be arranged in other ways, e.g., so that a side section or the upper section of the insertion portion 2 (the portion near the slot 6) extends distally further than other sections of the insertion portion 2. In addition, it will be understood that the angle at which the end face 5 is arranged relative to the passageway may be any suitable one, including 90 degrees, or perpendicular to the passageway. In this embodiment, the end face 5 has a slight convex curve as viewed in FIG. 3, but the end face may have a concave curve or be flat. The edges of the end face 5 may be rounded, softened or otherwise configured to aid in insertion of the speculum 1 either with or without an obturator 9. As shown in the bottom view in FIG. 4, the leading portion of the end face 5 may be somewhat flattened, or alternately may be more pointed or otherwise shaped as desired.

In one aspect of the invention, the speculum may include a handle portion with a gripping surface adapted to be gripped by a user to insert, rotate and otherwise manipulate the speculum 1. The gripping surface may extend proximally at a bottom side of the flange portion and have an arcuate shape that is approximately concentric with the wall of the insertion portion. A top side of the flange portion may be free of the handle portion so as to permit access to the passageway. (Terms “top,” “bottom” and “side” are used only in a relative sense herein for ease of description. However, the use of these terms should not limit the way in which the speculum may be used, including various orientations of the speculum in a body opening.) For example, as can be seen in FIGS. 5-7, the handle portion 32 extends proximally relative to the passageway 4 and defines an arcuate gripping surface that is approximately concentric with the wall of the insertion portion 2.

In accordance with one aspect of the invention, a gripping surface of the handle portion 32 may be relatively compact and may allow 360 degree rotation of the speculum 1 while in place in a body opening without interfering with the patient's legs or other body portions outside of the opening. For example, in one illustrative embodiment, the handle portion 32 may define an arcuate gripping region having a diameter or maximum chord length of 8 cm or less. In one aspect of the invention, the handle portion or other gripping region of the speculum may extend no further radially than any other part of the flange portion 3. As will be understood, the handle portion 32 may include ribs, serrations or other features to enhance a user's grip and may be otherwise arranged to function well ergonomically. In one embodiment, the illumination portion 8 may form all or part of a gripping region for a user, e.g., where the handle portion 32 in the illustrated embodiment is formed by a portion of the housing of the illumination portion 8.

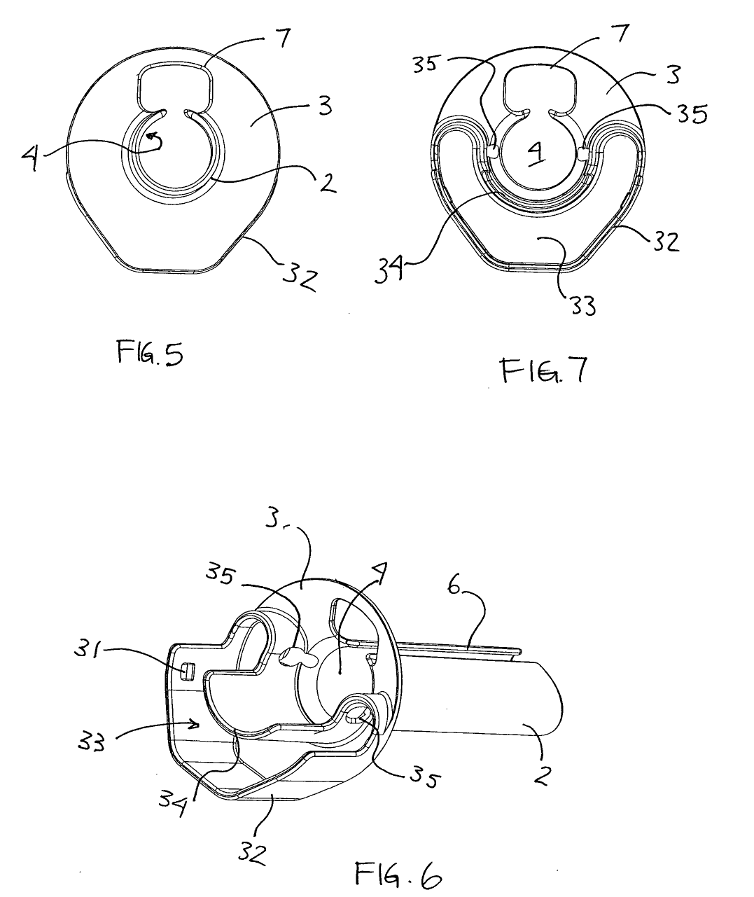

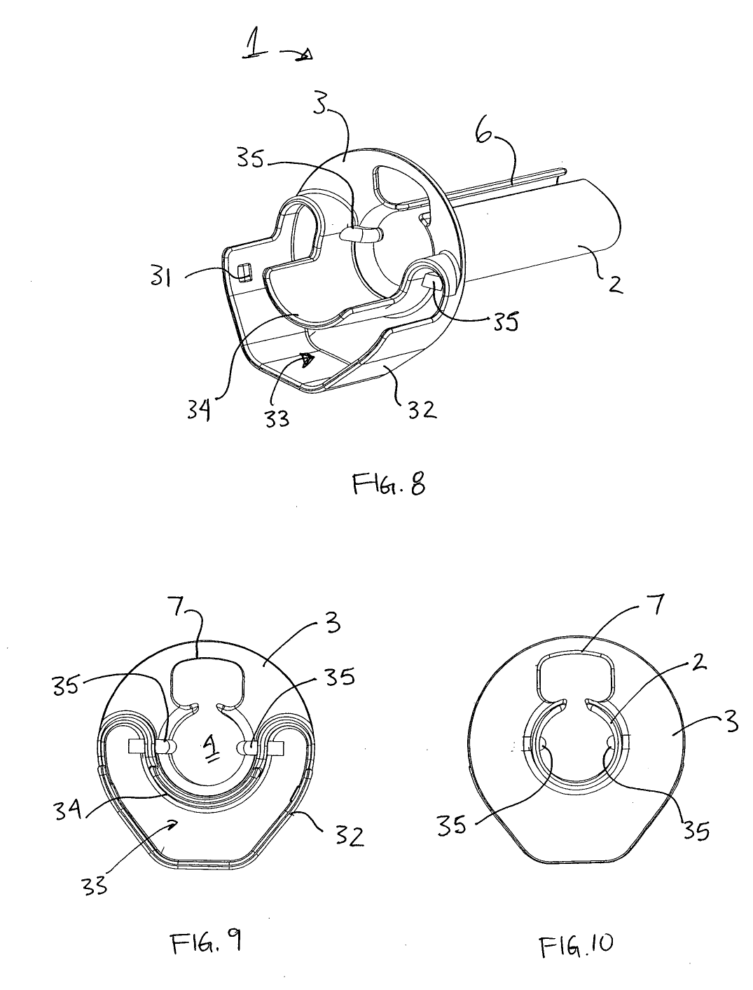

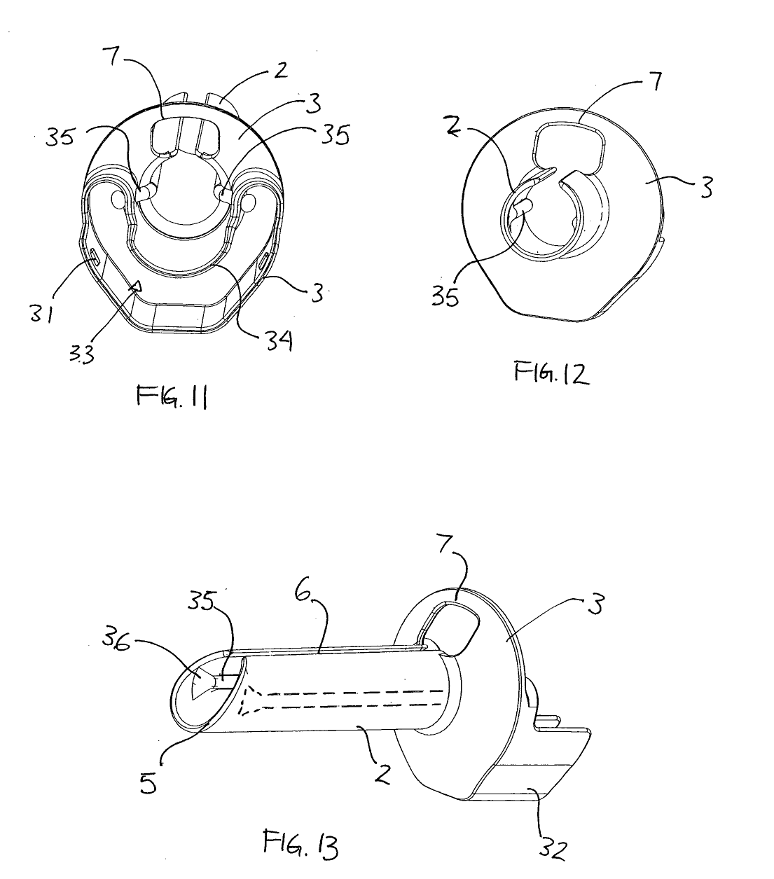

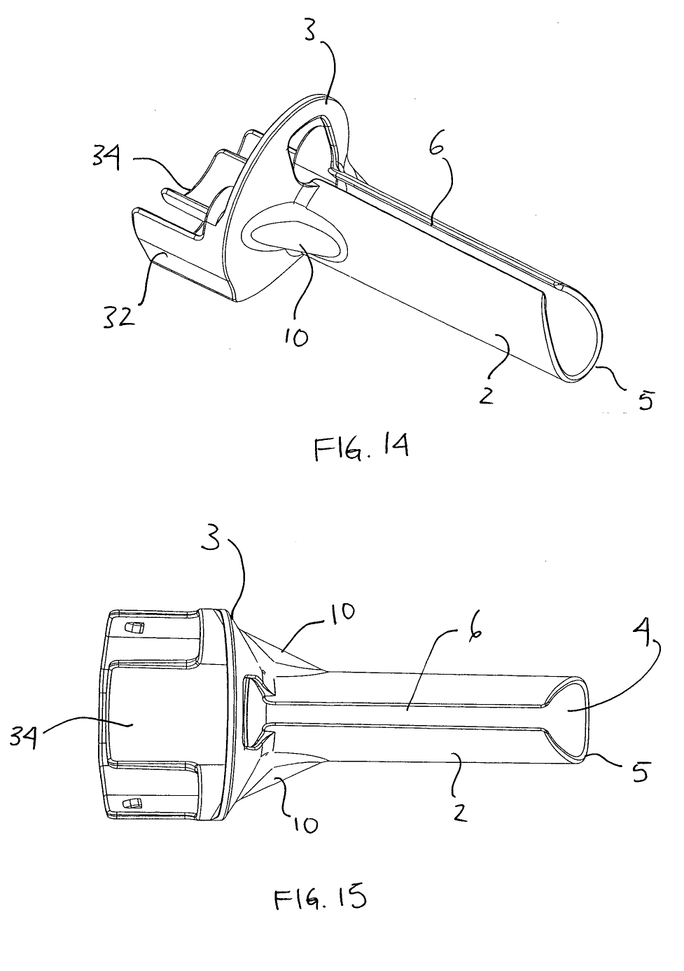

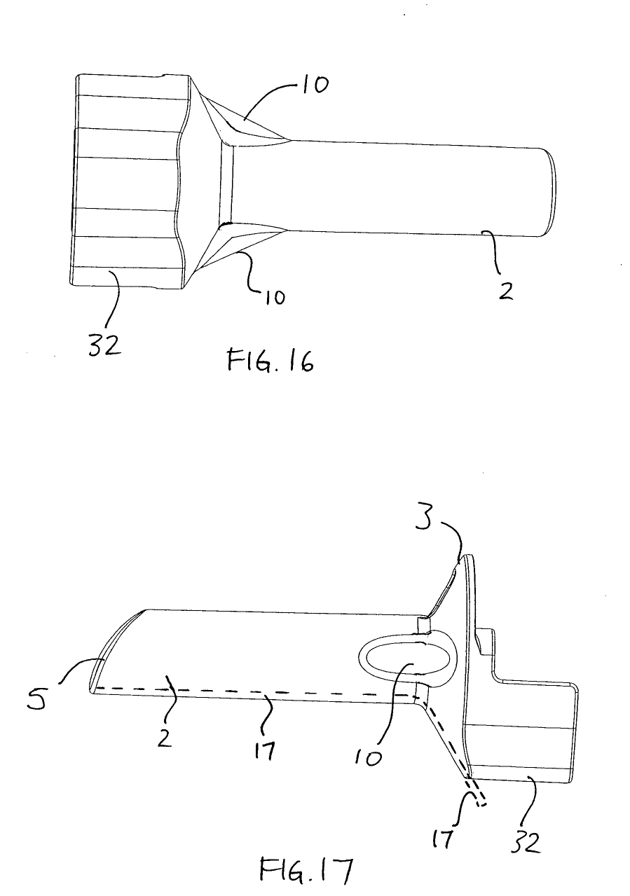

As illustrated in FIG. 6, in one aspect of the invention, the speculum 1 may include a recess 33 to receive the illumination portion 8. In this embodiment, the recess 33 may have an arcuate shape, as may be better seen in the rear view in FIG. 7. The recess 33 may be provided at a bottom side of the flange portion 3 so as to leave a relatively obstruction-free upper side at the flange portion 3 to allow easier access to the passageway 4. In one aspect of the invention, the flange portion 3 may include an extension 34 that communicates with the passageway 4 and provides a flow path for fluid in the passageway 4 to exit the insertion portion 2 and avoid contact with the illumination portion 8. When the speculum is used in vaginal applications, this feature may allow for the removal of fluids from the cervical area and/or uterus without the loss of fluids in the vagina. The extension 34 may also define part of the recess 33.

In this illustrative embodiment, the part of the flange portion 3 that forms the recess 33 for the illumination portion 8 includes one or more ports 35 that allow light emitted from the emitting regions 81 to be passed into and/or transmitted down the passageway 4. The ports 35 may be formed by holes or other opening arrangements in the flange portion 3, formed by transparent portions of the flange portion 3, and/or formed by a light pipe, optical fiber or other light transmitting structure, or other arrangement. The ports 35 may include a lens or other optical feature to focus, scatter, diffuse, filter or otherwise operate on the light emitted from the emitting regions 81. Alternately, the illumination portion 8 may include such optical features, e.g., at the light emitting regions 81. In this illustrative embodiment, the ports 35 allow light to enter the passageway 4 and be transmitted distally. The light may be transmitted by reflection or otherwise passing in the passageway and/or by transmission in the wall of the insertion portion or another structure, such as a light guide, optical fiber, or other. The light may be transmitted so as to illuminate a body portion at the distal end of the insertion portion 2 and/or other body portions along the length of the insertion portion 2. The wall of the insertion portion 2 may be coated or otherwise have a surface feature to reflect light internally down the passageway, if desired.

FIGS. 8-10 show a rear perspective view, a rear view and a front view, respectively, of another embodiment of a speculum 1. The speculum 1 in this embodiment is identical to the embodiment shown in FIGS. 1-7 except that the ports 35 in this embodiment include a light guide structure that transmits light from the illumination portion 8 to the insertion portion 2. Although two ports 35 are shown, any suitable number of ports 35 may be used in aspects of the invention. In this embodiment, the ports 35 include structures that receive light from the light emitting regions 81 and transmit the light (with some, little or no loss) into the wall of the insertion portion 2. Light transmitted by the ports 35 into the wall of the insertion portion 2 may be transmitted by the wall in a distal direction as discussed above. The ports 35 may alternatively, or additionally, be constructed and arranged to emit light into the passageway 4 so that the light may be transmitted down the passageway 4 toward the distal end. The ports 35 may be molded integrally with the flange and/or insertion portions 3, 2, or may be separate components that are assembled into the speculum 1. The ports 35 in this embodiment may be formed of a molded plastic, optical fiber or other suitable material. Input faces of the ports 35 may be arranged to receive light from the light emitting regions 81 of the illumination portion 8 to minimize light loss and/or direct the light appropriately into the port 35.

FIGS. 11-13 show a rear perspective view, a front perspective view and a side perspective view, respectively, of an illustrative embodiment in which light from the illumination portion 8 is transmitted distally by one or more light guides. In this embodiment, the ports 35 include extended light guides that extend at least partially down the passageway 4. The ports 35 may be molded unitarily with the insertion portion 2 and/or the flange portion 3, or may include separately made structures that are assembled with the speculum 1, e.g., by adhering the structures to the inner wall of the insertion portion 2. The ports 35 may emit light along their length, thereby providing illumination in the passageway 4, and/or may emit light at a distal end of the ports 35. In this embodiment, the ports 35 communicate with fan-shaped portions 36 that may serve to emit light in a desired way into the passageway 4 and/or transmit light into the wall of the insertion portion 2, e.g., for emission at the end face 5.

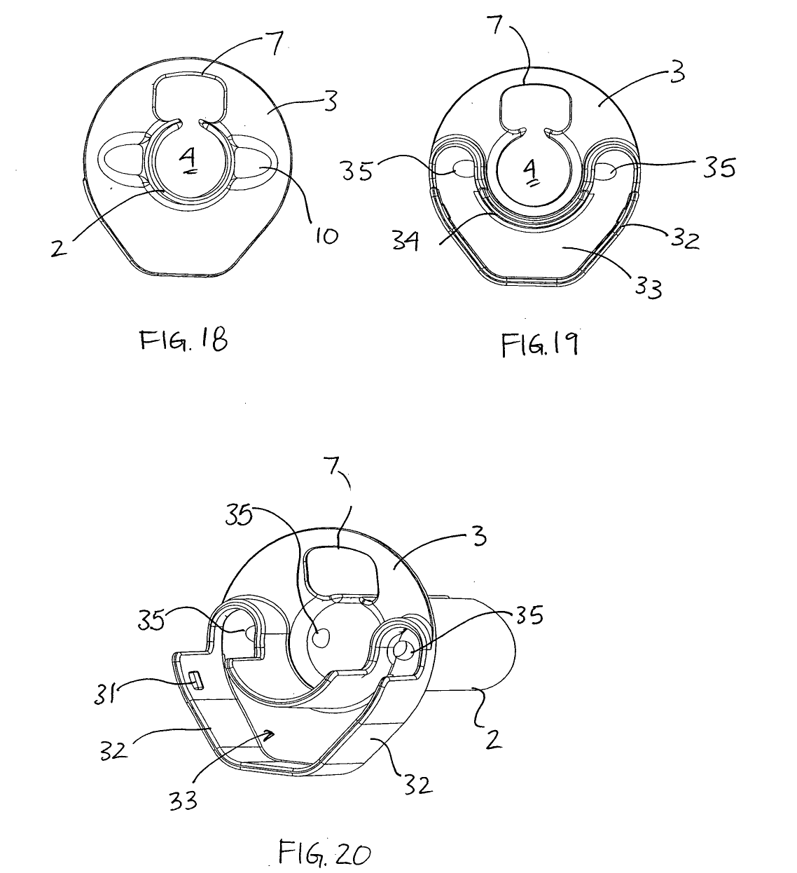

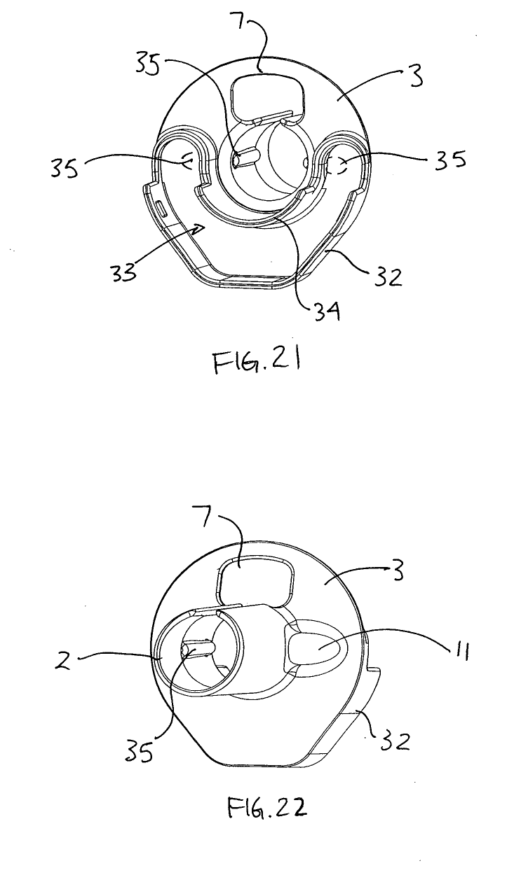

FIGS. 14-20 show another illustrative embodiment of a speculum in accordance with aspects of the invention. This embodiment is similar in many respects to that in FIGS. 1-7, and thus features that are the same are not described. In this illustrative embodiment, the speculum 1 includes two rib portions 10 that extend between the flange portion 3 and the insertion portion 2 and provide an enclosed region for the ports 35 (see FIGS. 19 and 20). Thus, the ports 35 allow for the emission of light into the passageway 4 and/or into the wall of the insertion portion 2 at a point that is more distally located than the arrangement in the FIGS. 1-7 embodiment. As with prior embodiments, the ports 35 may be formed by holes or openings in the flange portion 3 and/or the insertion portion as well as the ribs 10. The ports 35 may additionally or alternatively include light guide structures or other optical components that focus, transmit, diffuse, filter, disperse or otherwise operate on the light. For example, FIGS. 21 and 22 show an alternative to this embodiment in which the ports 35 are formed by transparent or other suitably arranged portions of the flange portion 3, the ribs 10 and the insertion portion 2 to transmit light distally. As in the embodiments above, the ports 35 may be formed by structures that are attached to or otherwise assembled with the speculum after the flange portion 3 and/or the insertion portion 2 are formed, or may be formed integrally with other portions of the speculum. In short, any suitable arrangement may be used to form ports that direct light from an illumination portion 8 to another portion of the speculum 1.

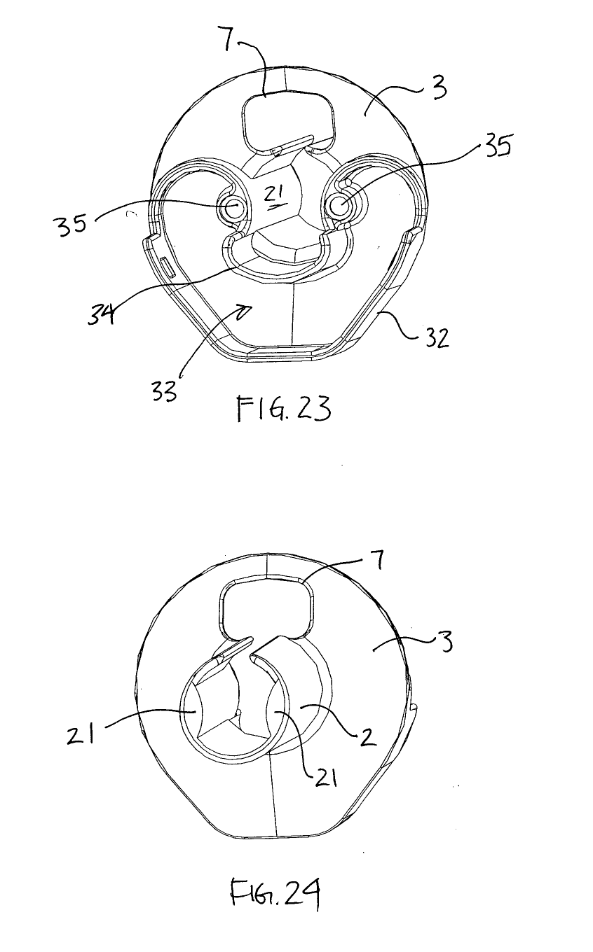

FIGS. 23 and 24 show rear and front perspective views, respectively, of another embodiment in which the ports 35 include thickened or bulging side sections 21 of the insertion portion 2 that transmit light toward the distal end. Light transmitted in the side sections 21 may be emitted at the distal end of the insertion portion 2 and/or at some intermediate location in the passageway, such as 50-80 mm from the distal end. Lenses or other features may be incorporated into the side section 21 so that light is emitted, focused, scattered, diffused, etc. into the passageway 4 in a way to suitably illuminate an area of interest. Diffusing of light may also occur by a coating or other feature on the internal surface of the insertion portion 2.

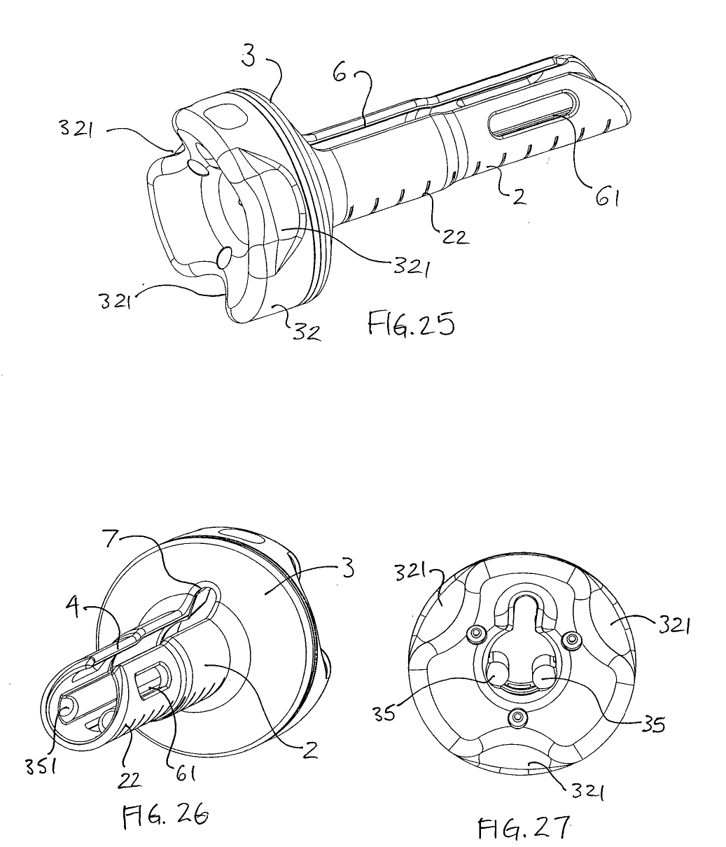

FIGS. 25-27 show side, front and rear perspective views, respectively, of another embodiment in which the handle portion 32 extends entirely around the passageway 4. The handle portion 32 also includes three relieved portions 321 that may aid in providing access to the passageway 4 and/or aid in helping a user better grip the speculum 1. In this embodiment, the insertion portion 2 also includes two openings 61 in the wall. As discussed above, these openings 61 may be provided in any size, shape, orientation, etc. This embodiment also includes ports 35 having bar-shaped members extending along an inner surface of the wall of the insertion portion 2. These bar-shaped members may include a lens or other feature 351 near the distal end of the insertion portion 2 to provide light to an area of interest. Also in this embodiment, at least part of the illumination portion 8 may be incorporated into the speculum 1, e.g., partially housed inside the handle portion 32 and capable of providing light to the ports 35. The insertion portion 2 also includes an optional reduction in diameter or cross-sectional size about midway along the length of the insertion portion 2. This step may be useful when accessing openings that have a reduced cross-sectional size at deeper regions as compared to outer portions of the opening. The change in cross-sectional size of the insertion portion 2 may be provided at any location and in any way, such as by having the insertion portion 2 gradually taper, e.g., forming a conical or frustoconical shape. Grooves, indicator markings or other features 22 may provide an indication as to the depth at which the insertion portion is placed in the opening.

Portions of the speculum 1, such as the insertion portion 2 and/or the flange portion 3, may be made to be disposable, for example being molded as a single unitary part of polycarbonate, crystal polystyrene or any other suitable material, or combination of materials, whether opaque, transparent or translucent. Portions of the speculum may be molded together in a unitary form, or may be made separately and joined together, e.g., by an adhesive, mechanical fasteners, welding, removable attachment, and so on. Various portions of the speculum may be made of any suitable material, such as plastics, composites, metals, and so on.

In addition, or alternately, portions of the speculum 1, such as the illumination portion 8, may be made to be reusable using, for example, a transparent plastic (e.g., at least for the illumination portion housing) suitable for cleaning with chemicals, gas, autoclave or microwave processing. For example, the illumination portion 8 may be made so that the entire device, including the housing, batteries, electronics, LEDs or other light emitting devices, etc., can be autoclaved or otherwise processed to sterilize, clean or otherwise make the illumination portion suitable for subsequent use in a medical procedure. Electronic components capable of withstanding such processing will be understood by one of skill in the art. One type of autoclavable battery that may be used in the illumination portion is a CR2 lithium battery made by Sanyo.

Portions of the speculum may be coated or treated in different ways, such as applying a charcoal or other coating to the interior wall of the insertion portion to help make the speculum more resistant to damage by laser or other light forms. Coatings or other treatments may be used to make the speculum more resistant to damage by heat, chemicals, abrasion, etc.

In one embodiment when using a speculum in accordance with the invention, the insertion portion 2 and obturator 9 may be arranged so the obturator 9 is inserted into the passageway 4 as shown in FIG. 2 and the speculum advanced into the vagina. Clear water soluble or other suitable lubricant may be placed along the outside of the speculum, e.g., to enhance visualization. Once the cervix is located (e.g., by visualizing the cervical os through a distal opening in the obturator 9), the obturator 9 may be pulled back from within the insertion portion 2 and be partly, or completely withdrawn. The insertion portion 2 may be rotated until the distal end 6 of the cervix is located and pulled into view. Rotation of the insertion portion 2 may take place with or without the obturator in place. Since the beveled end of the speculum 1 may increase the surface area of the end of the insertion portion 2, procedures such as a pap smear may be accomplished. A smaller speculum may thus be employed using this configuration because the sides of the speculum hold the lateral walls of the vagina from obstructing the view of the person during procedures such as a pap smear.

In addition, the obturator 9 need not include any lip, ring, pin or other feature that limits the depth to which the obturator 9 may be inserted into the insertion portion 2. Instead, the obturator 9 may be arranged so that it may be passed completely through the passageway 4. With such an arrangement, the obturator 9 may be first inserted into the body opening alone without the insertion portion 2. Thereafter, the insertion portion 2 may be slid over the proximal end of the obturator 9 and inserted into the opening while the obturator 9 remains in place.

In another aspect of the invention, the continuous sidewall configuration of the insertion portion may be an improvement over traditional specula having a bivalve configuration. The nearly continuous sidewall (with properly shaped and sized openings for, e.g., taking biopsies) may prevent vaginal or other tissue from moving into the line of sight and obscuring the examiner's view, thus improving the view down the longitudinal axis of the speculum. This contrasts with a traditional bivalve speculum, which has no continuous sidewalls to keep tissue from hindering the view down the longitudinal axis of the device. There is also the possibility that a vaginal examination may be performed in the lateral position instead of the dorsal lithotomy position because the side walls of the vagina are not obstructing the view.

As discussed above, different lighting arrangements may be used to help treat or visualize different conditions at the body opening. For example, illumination carried by the obturator 9 and/or insertion portion 2 (whether visual and/or non-visible light) may neutralize or prevent chemical changes that occur or may occur in the body opening, may neutralize harmful chemicals (such as lowering cytokines or inflammatory chemicals in a vagina), activate or enhance the action of photosensitive or photo-activated chemicals (such as peroxide chemicals), activate chemicals that form a barrier for viral, chemical or bacterial invasion, etc. The obturator 9 may be used as an applicator for medication, e.g., by introducing the medication through an opening at the distal end of the obturator, or by apply the medication to the distal end of the obturator while removed from the insertion portion, and then inserting the treated obturator into the insertion portion. For example, therapeutic use of light and/or activated creams/chemicals may be used to treat early onset of labor that may be caused by tumor necrosis factor which causes changes at the cervix.

The speculum may be available in various sizes to accommodate differences between patient anatomies, with a larger size available to also address procedural difficulties encountered in the operating room as well as the office setting. For example, the length of the insertion portion 2 may range from approximately 7-15 cm, while the length of the obturator 9 may range from approximately 8-16 cm. The outside diameter of the insertion portion 2 may range from approximately 1.5-4 cm.

Furthermore, the speculum may include a magnification component such as one or more lenses that allows for one or more magnification settings that may be used to facilitate examinations, e.g., enlarge the view of an area of interest in the passageway 4 for examination purposes.

In another aspect of the invention, the insertion portion 2 wall may have one or more openings in addition to, or in place of, the slot 6. These openings may be located anywhere along the length of the insertion portion 2 and inward from the proximal or distal end. The openings may have any suitable shape, such as oval, round or otherwise shaped, in various positions, making rotation of the instrument and procedures such as biopsy of the vaginal walls and cervix possible. In the embodiment where the rest of the insertion portion 2 is constructed of clear plastic, the use of an opening of a relatively small size may eliminate or limit the resultant blood loss from the area of a biopsy or other procedure, thus preventing obstruction of the view.

In one embodiment, even though the speculum in accordance with aspects of the invention may end up being slightly longer than a typical bivalve speculum, because light may be directed near the distal end and along the walls of the vagina, the instrument may not have to be inserted all the way up to the flange portion 3. On the other hand, a conventional speculum has to be inserted all the way to the handle end to minimize the need to open the blades any more than necessary. In a very short patient, there is a sensation of having very deep placement of the bivalve speculum to achieve this goal. An embodiment of the invention may be more versatile than a traditional bivalve speculum, which may require the doctor to use a much smaller speculum, such as a pediatric size, thereby potentially limiting the discomfort of a shorter patient. At the same time, the use of a smaller bivalve speculum may result in losing quality in terms of the ability to visualize and conduct procedures in the vagina, cervix and uterus. An aspect of the invention provides a more versatile speculum that may be used with patients of varied sizes, without losing any ability to conduct a thorough examination.

In another embodiment, the speculum 1 may be fitted with a minicam or other image capturing device for visualization and/or recording the appearance of the body opening being examined. The obturator may be fitted with an image capturing device near its distal end to visualize and record the appearance of the walls of the opening. Also, an image capturing device may be placed on a wand, or other device, and the walls of the body opening could be inspected in a 360 degree manner as the insertion portion of the speculum is withdrawn.

The speculum may also be arranged to provide different functions. For example, the insertion portion 2 may include one or more channels (such as that shown at reference number 11 in FIG. 17) that are used to evacuate smoke or fluids from the opening, or to provide a source of irrigation to the opening. The channels may be formed integrally with the insertion portion 2 or attached to the insertion portion 2. The channels may be arranged at any location on the speculum, such as at a bottom, side and/or top portion. One or more connection points may be provided to facilitate attachment of tubes or other sources of vacuum or irrigation fluid.

Specula arranged in accordance with aspects of the invention may be used in a variety of different ways, with a variety of different tools and a variety of different body openings. For example, the speculum may be used in procedures involving the use of a sharp toothed tenaculum, a long Allis clamp, crushing clamps, a scalpel or suturing instrument, forceps of different types, a curette, one or more dilators, hot loop endocervical wires, pap smear tools, a colposcope lens/light or other related device, one or more sponges, chemicals or other agents for treating different medical conditions, a pipette for sampling chorionic villous tissue, a fetal scalp electrode or fetal oxygenation detector, one or more catheters including balloon-tipped catheters and hysterosalpingogram catheters, intrauterine device insertion tools, vaginal pessaries, contraceptive diaphragms or other devices, intrauterine insemination catheters or embryo transfer catheters, ultrasound probes, a syringe, a biopsy needle, endometrial ablation devices, and others. Body openings that may be used with the specula include the vagina, rectum, mouth, throat, ear, a surgical opening, and so on. Such openings may be present in any animal, including human or other mammal, reptiles, avian, or other.

In another embodiment, a lighting feature, for example, LEDs may be built in, embedded or otherwise incorporated into any number of medical devices used for retraction in the body. Retractors of many shapes, sizes and configurations may benefit from incorporated light sources positioned along the instrument and/or placed at the working end of the instrument. Such retractors may be used in neurological, orthopedic, cardiac, urological, oral and other procedures known to one of skill in the art of medical instruments. Furthermore, other medical devices that may benefit from an incorporated light feature may include devices for cutting, cautery, gripping or grasping, probing, examining, suction, irrigation, drilling, fusion, suturing, as well as other devices known to those of skill in the art.

While the invention has been described with reference to various illustrative embodiments, the invention is not limited to the embodiments described. It is evident that many alternatives, modifications and variations of the embodiments described will be apparent to those of ordinary skill in the art. Accordingly, embodiments of the invention as set forth herein are intended to be illustrative, and not limiting the scope of the invention. Various changes may be made without departing from the scope of the invention.

1. A medical instrument for use in accessing an opening in a body, the opening including material around the opening, the instrument comprising:
an insertion portion constructed and arranged for insertion into the opening in the body, the insertion portion having an arcuate-shaped wall that extends from a proximal end to a distal end of the insertion portion, the wall defining an elongated passageway from the proximal end to the distal end and having a slot that extends along the entire length of the wall from the proximal end to the distal end; and
a flange portion having a section that forms a continuous band that is joined to the wall at the proximal end of the insertion portion and that extends radially outwardly away from the passageway, the flange portion including a notch in communication with the slot in the wall of the insertion portion;
wherein the insertion portion is sized to be inserted into the opening in the body with the flange portion positioned outside of the opening so that the insertion portion displaces at least some of the material at the opening, the passageway in the insertion portion providing access to the opening from the proximal end to the distal end of the insertion portion.

2. The instrument of claim 1, further comprising a handle portion with a gripping surface adapted to be gripped by a user for manipulation of the speculum, the gripping surface extending proximally from the flange portion and having an approximately arcuate shape that is approximately concentric with the wall of the insertion portion.

3. The instrument of claim 1, wherein the arcuate-shaped wall forms an approximately cylindrically-shaped barrel having a longitudinal axis, and the slot is formed along the longitudinal axis.

4. The instrument of claim 1, further comprising:
an obturator constructed and arranged to be inserted into the passageway of the insertion portion, the obturator having a distal end constructed and arranged to extend from the distal end of the insertion portion and facilitate insertion of the insertion portion into the opening in the body.

5. The instrument of claim 4, wherein the obturator includes an approximately cylindrical body having a tapered end with an opening formed at the distal end of the obturator.

6. The instrument of claim 1, wherein the insertion portion includes a beveled end at the distal end of the insertion portion.

7. The instrument of claim 6, wherein the wall forms an approximately cylindrically-shaped barrel having a longitudinal axis, wherein the slot is formed along the longitudinal axis, and wherein an end face of the wall at the distal end is arranged at an angle to the longitudinal axis and so that a length of the approximately cylindrically-shaped barrel is shortest between the proximal end and a portion of the wall near the slot.

8. The instrument of claim 1, further comprising an illumination portion, and wherein the insertion portion is constructed and arranged to direct light from the illumination portion toward the distal end of the insertion portion.

9. The instrument of claim 8, wherein the illumination portion is constructed and arranged to provide light to two or more separate portions of the insertion portion.

10. The instrument of claim 8, wherein the flange portion includes a recess that receives part of the illumination portion.

11. The instrument of claim 1, further comprising a handle portion constructed and arranged to be gripped by a physician to allow manipulation of the insertion portion.

12. The instrument of claim 11, wherein the handle portion has a maximum radial extent that is equal to or less than a maximum radial extent of any other section of the flange portion.

13. The instrument of claim 1, wherein the continuous band of the flange portion has an outer surface that has a frustoconical shape.

14. A medical instrument for use in accessing an opening in a body, the opening including material around the opening, the instrument comprising:
an insertion portion constructed and arranged for insertion into the opening in the body, the insertion portion having an arcuately-shaped wall that extends from a proximal end to a distal end of the insertion portion, the wall defining an elongated passageway having a longitudinal axis extending from the proximal end to the distal end and having a slot that extends along an entire length of the wall from the proximal end to the distal end, an end face of the wall at the distal end of the insertion portion being arranged at a non-perpendicular angle to the longitudinal axis; and
a flange portion having a portion that is joined to the wall at the proximal end of the insertion portion and that extends radially outwardly away from the passageway;
wherein the insertion portion is sized to be inserted into the opening in the body and displace at least some of the material with the flange portion positioned outside of the opening, the elongated passageway in the insertion portion providing access to the opening from the proximal end to the distal end of the insertion portion.

15. The instrument of claim 14, wherein the end face is arranged at an angle to the longitudinal axis so that a length of the insertion portion between the proximal end and a portion of the end face near the slot is shorter than a length of the insertion portion between the proximal end and a portion of the end face opposite the slot.

16. The instrument of claim 14, wherein the flange portion includes a notch in communication with the slot in the wall of the insertion portion.

17. The instrument of claim 14, wherein the flange portion has a section that forms a continuous band that is joined to the wall at the proximal end of the insertion portion.

18. The instrument of claim 14, further comprising a handle portion with a gripping surface adapted to be gripped by a user for manipulation of the speculum, the gripping surface extending proximally from the flange portion and having an approximately arcuate shape that is approximately concentric with the wall of the insertion portion.

19. The instrument of claim 14, wherein the arcuate-shaped wall forms an approximately cylindrically-shaped barrel having a longitudinal axis, and the slot is formed along the longitudinal axis.

20. The instrument of claim 14, further comprising:
an obturator constructed and arranged to be inserted into the passageway of the insertion portion, the obturator having a distal end constructed and arranged to extend from the distal end of the insertion portion and facilitate insertion of the insertion portion into the opening in the body.

21. The instrument of claim 20, wherein the obturator includes an approximately cylindrical body having a tapered end with an opening formed at the distal end of the obturator.

22. The instrument of claim 14, further comprising an illumination portion, and wherein the insertion portion is constructed and arranged to direct light from the illumination portion toward the distal end of the insertion portion.

23. The instrument of claim 22, wherein the illumination portion is constructed and arranged to provide light to two or more separate portions of the insertion portion.

24. The instrument of claim 22, wherein the flange portion includes a recess that receives part of the illumination portion.

25. The instrument of claim 14, further comprising a handle portion constructed and arranged to be gripped by a physician to allow manipulation of the insertion portion.

26. The instrument of claim 25, wherein the handle portion has a maximum radial extent that is equal to or less than a maximum radial extent of any other section of the flange portion.

27. A medical instrument for use in accessing an opening in a body, the opening including material around the opening, the instrument comprising:
an insertion portion constructed and arranged for insertion into an opening in a body, the insertion portion having a wall having an approximately arcuate cross-section that extends from a proximal end to a distal end of the insertion portion, the wall defining an elongated passageway having a longitudinal axis extending from the proximal end to the distal end;
a flange portion having a portion that is joined to the wall at the proximal end of the insertion portion and that extends radially outwardly away from the passageway, the flange portion having a handle portion with a gripping surface adapted to be gripped by a user for manipulation of the speculum, the gripping surface extending proximally at a bottom side of the flange portion and having an arcuate shape that is approximately concentric with the wall of the insertion portion, a top side of the flange portion being free of the handle portion so as to permit access to the passageway;
wherein the insertion portion is sized to be inserted into an opening in a body and displace at least some of the material with the flange portion positioned outside of the opening, the elongated passageway in the insertion portion providing access to the opening from the proximal end to the distal end of the insertion portion.

28. The instrument of claim 27, further comprising an illumination portion, and wherein the insertion portion includes at least one port that transmits light from the illumination portion toward the distal end of the insertion portion.

29. The instrument of claim 27, further comprising an illumination portion, and wherein the illumination portion is removably attached to the flange portion.

30. The instrument of claim 27, further comprising an illumination portion, and wherein the flange portion has a recess at a bottom side of the flange portion that receives the illumination portion.

31. The instrument of claim 27, further comprising an illumination portion that provides light to at least a portion of the insertion portion.

32. The instrument of claim 27, wherein the wall of the insertion portion includes a slot that extends along a length of the wall.

33. The instrument of claim 27, further comprising an extension that extends from the passageway and provides a path for fluid in the passageway to exit the insertion portion.

34. The instrument of claim 27, wherein the arcuate-shaped wall forms an approximately cylindrically-shaped barrel having a longitudinal axis, and a slot is formed in the wall along the longitudinal axis.

35. The instrument of claim 27, further comprising:
an obturator constructed and arranged to be inserted into the passageway of the insertion portion, the obturator having a distal end constructed and arranged to extend from the distal end of the insertion portion and facilitate insertion of the insertion portion into the opening in the body.

36. The instrument of claim 27, wherein the insertion portion includes a beveled end at the distal end of the insertion portion.

37. The instrument of claim 36, wherein the wall forms an approximately cylindrically-shaped barrel having a longitudinal axis, wherein a slot is formed along an entire length of the insertion portion, and wherein an end face of the wall at the distal end is arranged at an angle to the longitudinal axis and so that a length of the approximately cylindrically-shaped barrel is shortest between the proximal end and a portion of the wall near the slot.

38. The instrument of claim 27, wherein the flange portion includes a continuous band that extends radially from the proximal end of the insertion portion.
